(19) United States
(12) Patent Application Publication
(10) Pub. No.: US 2007/0093892 A1
(43) Pub. Date: Apr. 26, 2007

(54) Maintaining preoperative position of the posterior lens capsule after cataract surgery

(75) Inventor: Richard J. Mackool, Astoria, NY (US)

Correspondence Address: ALCON, IP LEGAL, TB4-8, 6201 SOUTH FREEWAY, FORT WORTH, TX, 76134, US

(73) Assignee: Alcon Manufacturing, Ltd.

(21) Appl. No.: 11/254,945

(22) Filed: Oct. 20, 2005

(51) Int. Cl. A61F 2/16

(52) U.S. Cl. 623/6.42; 623/6.38

(57) ABSTRACT

Intraocular lens implant that includes a lens optic and lens haptics configured to maintain a preoperative position of the posterior lens capsule after cataract removal and insertion of a lens implant. The lens haptics have proximal and distal portions, with the distal portions lying in a common plane and the lens optic extending in a lens optic plane. The distance between the planes may be at least substantially the same dimension as or larger than a shift distance that the posterior lens capsule would otherwise traverse between its normal anatomical location and its shifted anatomical location where it not constrained. The shifted anatomical location arises naturally after both removal of cataract lens material and removal of a portion of an anterior capsule.

### BACKGROUND OF THE INVENTION

The present invention relates to an apparatus, use and method that maintains a preoperative position of the posterior lens capsule during and after cataract removal and insertion of a lens implant.

A lens haptic conventionally is configured to either be in the plane of the lens optic or to encounter the plane of the lens optic at an angle of 10 degrees or less. The purpose for the lens haptic is to attain stable lens fixation.

The present inventor, an experienced surgeon who has performed tens of thousands of cataract operations, was quoted in the May 2004 edition of Ophthalmology Management as follows with respect to Cataract Surgery: Managing Weak Zonules and an anterior capsule support system bearing his name is described. Such an anterior capsule support system is described in U.S. Pat. No. 6,183,480 by the same inventor.

The Anterior Chamber Lens Option: Richard J. Mackool, M.D., director of The Mackool Eye Institute and Laser Center in Astoria, N.Y., and senior attending surgeon at the New York Eye and Ear Infirmary, notes that being able to put an IOL intraocular lens) in the bag with the endocapsular ring doesn't mean you always should. “When you know that a weak zonule could lead to eventual dislocation of the capsular bag and its IOL,” Dr. Mackool says, “consider placing an endocapsular ring in the bag to establish a (hopefully) permanent position for the capsule—but implant an anterior chamber lens instead of placing the IOL in the bag or sulcus. The capsule will separate the anterior and posterior segments, reducing the likelihood of macular edema and vitreoretinal complications; meanwhile, the anterior chamber lens will remain perfectly fixated.”

The Mackool Cataract Support System. A capsular tension ring may not hold the bag in place during phaco if the zonule is especially weak. Surgeons sometimes use iris retractors on the rim of the capsulorhexis to try to manage this problem, but iris retractors aren't designed for this purpose; their hooks have a short return, and if the retractor comes in at an angle, the hook is not in the plane of the rim you're trying to grab.

In contrast, the Cataract Support System designed by Richard Mackool, M.D., has a hook return of 2.5 mm, and the hook is angled to be in the plane of the anterior capsule. As a result, it's easy to get a firm and reliable grip on the rim. An endocapsular ring can be inserted before the Cataract Support System is removed.

The Mackool system: maintains the position of the capsule during phaco; supports the fornix as well as the capsulorhexis rim so the peripheral capsule isn't attracted toward the phaco tip; avoids the problem of the endocapsular ring trapping cortex; prevents zonular stress during nuclear rotation; makes it possible to perform posterior chamber phaco even with dislocated cataracts having almost no zonular support.

Conventional intraocular lenses have angulated attachments, otherwise known as haptics, but their purpose is to keep the body of the lens implant, also known as the lens optic, away from the iris. Such conventional intraocular lenses with haptics attachment, however, are not designed to keep the posterior lens capsule from undergoing a forward (anterior) shift after cataract surgery.

The present inventor recognizes that the forward (anterior) shift of the posterior lens capsule after cataract surgery is probably the cause, although indirectly, of the increased risk of retinal detachment after cataract removal. It would be desirable to prevent such a forward (anterior) shift.

### BRIEF SUMMARY OF THE INVENTION

One aspect of the invention resides in an intraocular lens implant with a lens optic and lens haptics configured to maintain a preoperative position of the posterior lens capsule during and after cataract removal and insertion of a lens implant. The lens haptics have proximal and distal portions, with the proximal portions being closer to the lens optic than are the distal portions. The lens optic extends in a lens optic plane and the distal portions of the lens haptics are in a common plane. The two planes are separated from each other by separation distance. That separation distance is preferably at least substantially the same dimension as or larger than a shift distance that the posterior lens capsule would otherwise traverse between its normal anatomical position and its shifted position if not constrained. The shifted position arises naturally after both removal of lens material within a lens capsule and removal of a portion of an anterior capsule.

### BRIEF DESCRIPTION OF THE DRAWINGS

For a better understanding of the present invention, reference is made to the following description and accompanying drawings, while the scope of the invention is set forth in the appended claims.

FIG. 1 is a schematic of the eye immediately after cataract removal.

FIG. 2 is a schematic of the eye with an angulated lens haptic of an embodiment of the present invention in position to prevent the posterior lens capsule from shifting forward.

FIG. 3. is a schematic of the eye with a right angled lens haptic of a further embodiment of the present invention in position to prevent the posterior lens capsule from shifting forward.

FIG. 4 is a schematic of the eye with a curved lens haptic of another embodiment of the present invention in position to prevent the posterior lens capsule from shifting forward.

FIG. 5 is a schematic of the eye with a deformable, soft lens haptic and two stiff rod-like structures in accordance with an additional embodiment in position to prevent the posterior lens capsule from shifting forward.

FIG. 6 is a schematic of the eye with a structure in accordance with yet additional embodiment in position to maintain a position of a lens implant.

FIG. 7 is a schematic representation of an interrupted, non-continuous ring suited to use as the structure in the embodiment of FIG. 6.

FIG. 8 is an enlargement of a portion of FIG. 6 for purposes of clarity.

### DETAILED DESCRIPTION OF. THE INVENTION

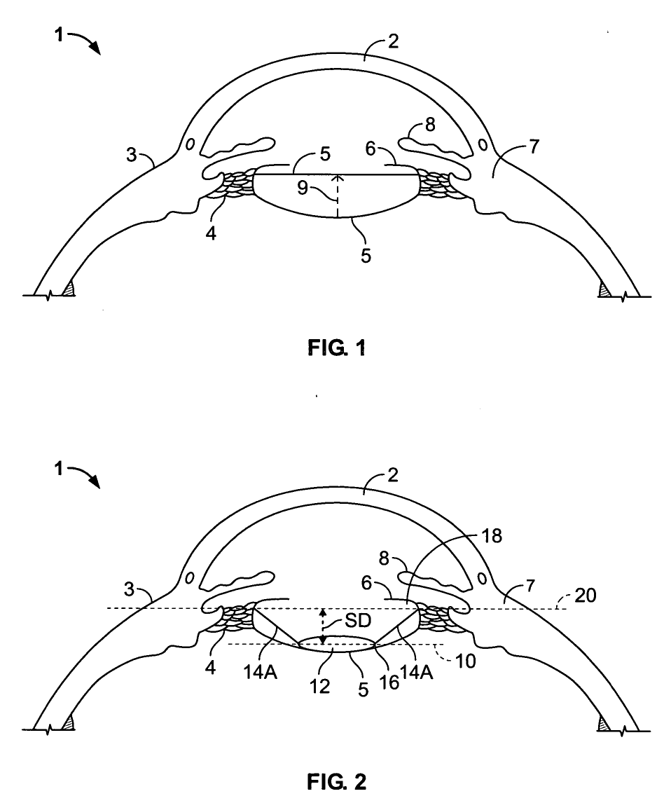

Turning to FIG. 1, eye 1 is shown with a cornea 2, sclera 3, zonule 4, posterior lens capsule 5 before and after the shift, anterior lens capsule 6, cilary body 7 and iris 8 in their anatomically correct locations immediately after removal of the cataract from cataract surgery. The cataract lens material and portion of the anterior lens capsule 8 are absent after cataract surgery. The position of the posterior lens capsule 5 is indicated in its normal anatomic location prior to cataract surgery (neighboring the base of direction arrow 9) and the position of the posterior lens capsule is indicated following cataract removal (neighboring the arrow point of the direction arrow 9).

Under conventional surgical techniques and implantation methods, the natural forward (anterior) shift distance that the posterior lens capsule traverses after cataract surgery when left unconstrained is 2-5 millimeters and is often 3-4 millimeters. The direction of the forward shift is anteriorly toward the cornea in the direction indicated by the direction arrow 9 as shown in FIG. 1. If a lens implant is not inserted, the forward (anterior) shift of the posterior lens capsule will exceed the several millimeter shift after cataract surgery that results when conventional lens implantation techniques are used that do not prevent the forward shift of the posterior lens capsule.

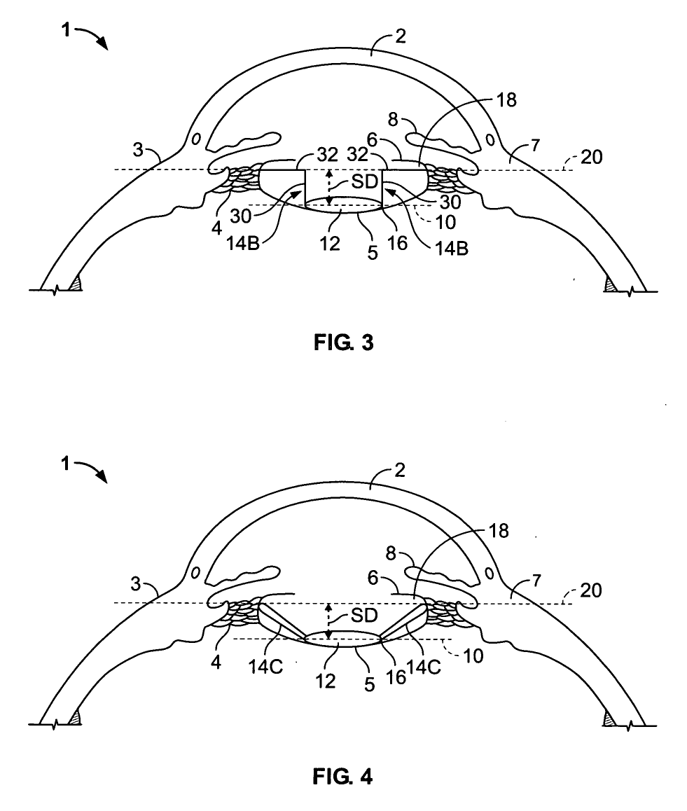

FIGS. 2 and 3 show an intraocular lens implant within the eye 1 that includes a lens optic 12 and lens haptics configured so that the optic of the implant holds the posterior lens capsule in its normal position. In the case of the embodiment of FIG. 2, the lens haptics 14A have a markedly angulated configuration. As such, each forms a significantly larger angle at the haptic-optic junction than conventional lens haptics that are designed to either be in the plane 10 of the lens optic or to encounter the plane 10 of the lens optic 12 at a 10 degree angle or less. In the case of the embodiment of FIG. 3, the lens haptic 14B has as much as 90 degrees angulation between portions 30, 32. As may be appreciated, horizontal contractile forces of the lens capsule after surgery in a central direction are obviated by the horizontal plane of the anterior most portion of the lens haptic of the embodiment of FIG. 3. The embodiments of FIGS. 2 and 3 are non-curved.

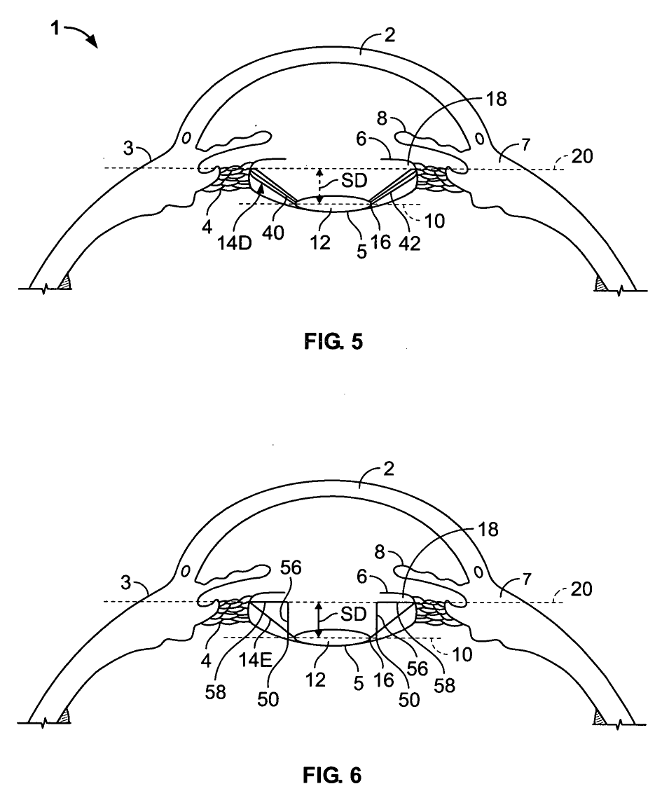

The embodiment of FIG. 4 reveals the intraocular lens with a lens optic 12 and lens haptics 14C being curved along the posterior lens capsule. The embodiment of FIG. 5 includes a lens optics 12 with a soft haptics 42 that is deformable, making it easier to insert the lens into the lens capsule than is the case for a non-deformable hard haptics. The embodiment of FIG. 5 also includes two stiff rod-like structures 40 that engage the lens optic 12 and thereby act as struts that prevent deformation of the soft lens haptics 42 over time, again as a result of contractile forces of the lens capsule.

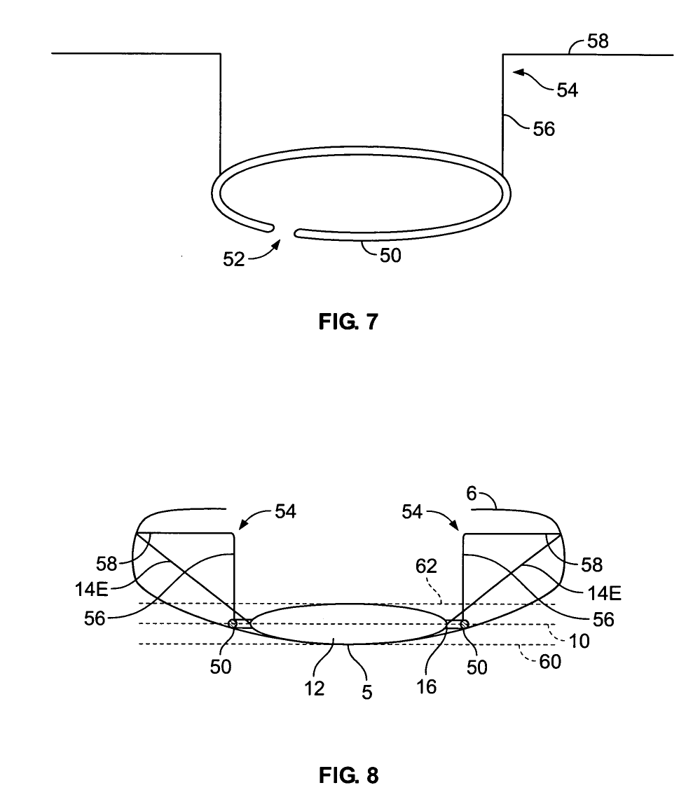

The embodiment of FIG. 6 shows a lens optic implant maintained in a desired position within the eye by a lens haptics 14E that is inserted after the implant is inserted. A structure 50 (FIGS. 6-8) that resembles a non-continuous ring with gap 52 is employed to help prevent the forward shift of the posterior lens capsule. Such a structure 50 is preferably incomplete, i.e., provides for the gap 52, to permit its collapse during insertion through an incision. The structure 50 may be made of a bendable material such as a biocompatible flexible metal having bendable properties so that the structure 50 may be bent from a collapsed condition into the non-collapsed condition of FIG. 7.

Preferably, the structure 50 is held in position by haptics 54, exemplified by right angled portions 56, 58, although such a haptics 54 may instead be inclined or curvilinear. For convenience, the structure 50 may be joined to the lens haptics 14E, although it may be arranged independent and separate from the lens haptics 14E. The haptics 54 may be made of the same material as the lens haptics 14E.

The structure 50 preferably has a larger diameter than the diameter of the lens optic implant 5 and lies in the same equator as the lens optic implant 5. That is, the structure 50 resides between the same two parallel planes 60, 62 that are separated from each other by a depthwise dimension of the lens optic implant 5 (see FIG. 8). When so situated in the same equator as the lens optic implant 5, the structure 50 is placed to help resist forces imposed by the posterior lens capsule that tend to otherwise cause the forward shift to occur.

Alternatively, the structure 50 may have the same or a smaller diameter than the diameter of the lens optic implant 5 in which case it would be arranged to be against the lens optic implant 5 to provide additional resistance against forces imposed by the posterior lens capsule that are responsible for the forward shift to otherwise occur.

Each of the embodiments of FIGS. 2-8 have common characteristics. Each reveals an intraocular lens implant that includes a lens optic 12 and lens haptics (14A, 14B, 14C, 14D or 14E as applicable). The lens optic 12 may be made of any biocompatible material used in a conventional lens optic, such as polymethylmethacrylate, hydrogels, soft acrylics, urethanes, and combination of such materials. The lens optic 12 may be foldable.

The lens haptics (14A, 14B, 14C, 14D or 14E as applicable) are fixed to the lens optic 12 preferably with their proximal portion 16 along or within the confines of the lens optic 12 yet outside the field of vision of the lens optic 12 and with their distal portion 18 projecting beyond the confines of the lens optic 12. The lens haptics (14A, 14B, 14C, 14D or 14E as applicable) may be bonded or physically fit to the lens optic 12. The proximal portions 16 are closer to the lens optic plane 10 than are the distal portions 18. The lens haptics (14A, 14B, 14C, 14D or 14E as applicable) may be made of a polymer or metallic substance to be substantially rigid and may be made of the same material as the lens optic 12.

Preferably, the distal portions 18 are in a common plane 20 that is separated from the plane 10 of the lens optic by a separation distance SD. That separation distance SD is at least substantially the same as, although preferably greater than, the shift distance that the posterior lens capsule 5 would traverse normally (if left unconstrained after cataract removal) to its greatest extent as indicated by the direction arrow 9 in FIG. 1. That is, the shift is between its normal anatomical position (prior to cataract surgery) and its shifted position. Thus, the posterior lens capsule 9 is not afforded the opportunity to effect a shift anteriorly.

The shift distance is an extent of a shift anteriorly that arises after conventional cataract surgery if no steps are taken to constrain the posterior lens capsule, because a portion of the anterior capsule (e.g., a central portion) and the cataract lens material are removed during the course of cataract surgery. The distal portions 18 may be free ends at which the lens haptics terminate. Alternatively, the distal portions 18 may be bends that allow the lens haptics to double back so that the free ends that are beyond the bends are closer to the lens optic plane than are the bends (distal portions).

The lens optic implant 5 with haptics 14A-14E of the invention may be implanted in the same manner as is known conventionally for conventional lens optic implants with haptics, i.e., through an incision and handled by forceps or like instruments. The lens optic implant 5 with haptics 14A-14D of FIGS. 2-5, when implanted, preferably prevents any shift forward of the posterior lens capsule after cataract surgery by any amount. The lens optic implant 5 with haptics 14E of FIGS. 6-8 when implanted, also helps prevent any shift forward of the posterior lens capsule after cataract surgery by any amount but does so in conjunction with the structure 50.

The present inventor recognized the need to prevent any amount of shift forward of the posterior lens capsule. The present invention permits the position of the posterior lens capsule, where it is prior to cataract surgery, to remain in the same position or largely so after cataract removal. The present invention may be in the form of an implant as previously described and/or as a device whose purpose is to keep the posterior lens capsule constrained against undergoing a forward (anterior) shift after cataract removal. Such an implant and/or device should be sterile prior to insertion into the eye.

The lens haptics 14A-14E should be relatively rigid, i.e., they should be rigid enough to resist deformation caused by capsular contraction as such deformation could conceivably cause them to lose their structure and fail to maintain the pre-operative position of the posterior lens capsule. Capsular contraction normally occurs along the axis of the lens haptics, and such contraction would have either no effect or an effect to further displace the optic in a posterior direction (toward the retinal surface). Such displacement would be resisted by the ocular components that are posterior to the posterior capsule (these non-compressible components consist of the vitreous body and wall of the eye, particularly the sclera). The contraction could, however, cause the haptics to impinge centrally on the visual axis and this would be undesirable.

The diameter of the capsular sac is remarkably similar from patient to patient. Therefore, if a line were drawn from the distal tip of one haptic to the distal tip of the other, it would probably be desirable for this dimension to be approximately 12-14 mm. It is unknown as to whether or not the function of the lens would be significantly altered by such a variation in dimension, but the present inventor suspects that it would not. The more important dimension is the distance between the plane of a line drawn between the distal tips of the two haptics (this line would be in a plane that was parallel to the lens optic), and the plane of the lens optic itself.

It is unknown as to whether or not one size lens haptics will fit all patients. As stated above, it is likely that a 12-14 mm distance between the tips of the two haptics would be the desirable size. As a corollary to this, endocapsular tension rings are horseshoe shaped plastic rings that are used to prevent capsular contraction after cataract surgery in eyes that are susceptible to this phenomenon. They are available in diameters between 12-14 mm, and the present inventor has used hundreds of these over the years. To date, the present inventor is unable to discern a clinical difference between the function of an endocapsular ring regardless of whether its diameter is 12, 13 or 14 mm. This does not mean that such a difference can not be discerned with a lens of the present invention, but it does indicate that contraction and distortion of the lens capsule generally compensates for the variations in size of various intraocular lenses and endocapsular tension rings.

While the foregoing description and drawings represent the preferred embodiments of the present invention, it will be understood that various changes and modifications may be made without departing from the scope of the present invention.

What is claimed is:

1. An apparatus useful in maintaining a position of a posterior lens capsule so as to prevent an anterior shift of the posterior lens capsule, comprising:
an implant that includes an intraocular lens that has a lens optic extending within a lens optic plane; and attachments including lens haptics with proximal and distal portions, the proximal portions being closer to opposite sides of the lens optic than are the distal portions, the distal portions being arranged within a common plane located out of the lens optic plane so that a separation distance is defined between the lens optic plane and the common plane that is substantially the same as or larger than a shift distance that would otherwise naturally occur by a posterior lens capsule within an adult eye between a normal anatomic location prior to cataract removal and a shifted anatomic location after removal of both cataract lens material and a portion of an anterior capsule, the lens haptics being sufficiently rigid to resist deformation caused by forces acting upon the lens haptics that are of a same magnitude and direction as that imparted by capsular contraction forces of the posterior lens capsule within the adult eye to traverse the shift distance.

2. An apparatus of claim 1, wherein the lens haptics have a configuration selected from a group consisting of angulation that defines an angle with the lens optic plane that is in excess of 10 degrees, angulation that defines a right angle with the lens optic plane, curvature, and deformable, soft haptics with two rigid rod structures arranged and configured to prevent deformation of the deformable, soft haptics over time.

3. An apparatus of claim 1, further comprising a structure engaging the lens optic and secured to the lens haptics.

4. An apparatus of claim 3, wherein the structure has an interrupted ring configuration having two free ends separated from each other by a gap.

5. An apparatus of claim 1, wherein the separation distance is selected from a group consisting of being greater than the shift distance and being at least approximately 2 mm.

6. Use of an implant for manufacture of an apparatus to maintain a position of a posterior lens capsule so as to prevent an anterior shift of the posterior lens capsule, comprising using an implant that includes an intraocular lens that has a lens optic extending within a lens optic plane, the attachments including lens haptics with proximal and distal portions, the proximal portions being closer to opposite sides of the lens optic than are the distal portions, the distal portions being arranged within a common plane located out of the lens optic plane so that a separation distance is defined between the lens optic plane and the common plane that is substantially the same as or larger than a shift distance that would otherwise naturally occur by a posterior lens capsule within an adult eye between a normal anatomic location prior to cataract removal and a shifted anatomic location after removal of both cataract lens material and a portion of an anterior capsule, the lens haptics being sufficiently rigid to resist deformation caused by forces acting upon the lens haptics that are of a same magnitude and direction as that imparted by capsular contraction forces of the posterior lens capsule within the adult eye to traverse the shift distance.

7. Use of claim 6, wherein the lens haptics have a configuration selected from a group consisting of angulation that defines an angle with the lens optic plane that is in excess of 10 degrees, angulation that defines a right angle with the lens optic plane, curvature, and deformable, soft haptics with two rigid rod structures arranged and configured to prevent deformation of the deformable, soft haptics over time.

8. Use of claim 6, further comprising a structure engaging the lens optic and secured to the lens haptics.

9. Use of claim 8, wherein the structure has an interrupted ring configuration having two free ends separated from each other by a gap.

10. Use of claim 6, wherein the separation distance is selected from a group consisting of being greater than the shift distance and being at least approximately 2 mm.

11. A method of assembling an apparatus useful in maintaining a position of a posterior lens capsule so as to prevent an anterior shift of the posterior lens capsule, comprising:
extending a lens optic of the implant within a lens optic plane; and
arranging the proximal portions of lens haptics of the attachments closer to opposite sides of the lens optic than are distal portions of the lens haptics, arranging the distal portions within a common plane located out of the lens optic plane so that a separation distance is defined between the lens optic plane and the common plane that is substantially the same as or larger than a shift distance that a posterior lens capsule within an adult eye would traverse naturally without being constrained between a normal anatomic location prior to cataract removal and a shifted position caused by both removal of cataract lens material and removal of a portion of an anterior capsule, the lens haptics being sufficiently rigid to resist deformation caused by forces acting upon the lens haptics that are of a same magnitude and direction as that imparted by capsular contraction forces of the posterior lens capsule within the adult eye to traverse the shift-distance.

12. A method of claim 11, further comprising configuring the lens haptics to have a configuration selected from a group consisting of angulation that defines an angle with the lens optic plane that is in excess of 10 degrees, angulation that defines a right angle with the lens optic plane, curvature, and deformable, soft haptics with two rigid rod structures arranged and configured to prevent deformation of the deformable, soft haptics over time.

13. A method of claim 12, further comprising engaging a structure with the lens optic and securing the structure to the lens haptics.

14. A method of claim 13, further comprising configuring the structure to have an interrupted ring configuration with two free ends separated from each other by a gap.

15. A method of claim 11, wherein the separation distance is selected from a group consisting of being greater than the shift distance and being at least approximately 2 mm.

16. An apparatus useful in maintaining a position of a posterior lens capsule so as to prevent an anterior shift of the posterior lens capsule, comprising an implant with attachments configured and arranged to secure the implant in a position that is spaced from an iris and that provide resistance to the posterior lens capsule undergoing a shift anteriorly to traverse a shift distance that would otherwise naturally occur within an adult eye between a normal anatomic location prior to cataract removal and a shifted anatomic location after removal of both cataract lens material and a portion of an anterior capsule, the lens haptics being sufficiently rigid to resist deformation caused by forces acting upon the lens haptics that are of a same magnitude and direction as that imparted by capsular contraction forces of the posterior lens capsule within the adult eye to traverse the shift distance.

17. Use of an implant for manufacture of an apparatus to maintain a position of a posterior lens capsule so as to prevent an anterior shift of the posterior lens capsule, comprising
using an implant with attachments to secure the implant in a position that is spaced from an iris and that provide resistance to the posterior lens capsule undergoing a shift anteriorly to traverse by a shift distance that would otherwise naturally occur by a posterior lens capsule within an adult eye between a normal anatomic location prior to cataract removal and a shifted anatomic location after removal of both cataract lens material and a portion of an anterior capsule, the lens haptics being sufficiently rigid to resist deformation caused by forces acting upon the lens haptics that are of a same magnitude and direction as that imparted by capsular contraction forces of the posterior lens capsule within the adult eye to traverse the shift distance.

18. A method of assembling an apparatus useful in maintaining a position of a posterior lens capsule so as to prevent an anterior shift of the posterior lens capsule, comprising
configuring and arranging an implant with attachments that secure the implant in a position that is spaced from an iris and that provide resistance to the posterior lens capsule undergoing a shift anteriorly to traverse a shift distance that would otherwise naturally occur by a posterior lens capsule within an adult eye between a normal anatomic location prior to cataract removal and a shifted anatomic location after removal of both cataract lens material and a portion of an anterior capsule, the lens haptics being sufficiently rigid to resist deformation caused by forces acting upon the lens haptics that are of a same magnitude and direction as that imparted by capsular contraction forces of the posterior lens capsule within the adult eye to traverse the shift distance.

19. An apparatus useful in maintaining a position of a posterior lens capsule so as to prevent an anterior shift of the posterior lens capsule, comprising:
an implant that includes an intraocular lens that has a lens optic extending within a lens optic plane, the implant having a depth separating two parallel planes from each other;
a structure arranged outside of the implant and between the two parallel planes;
haptics extending from the structure and having proximal and distal portions, the proximal portions being closer to opposite sides of the structure than are the distal portions, the distal portions being arranged within a common plane located out of the lens optic plane so that a separation distance is defined between the structure and the common plane that is substantially the same as or larger than a shift distance that would otherwise naturally occur by a posterior lens capsule within an adult eye between a normal anatomic location prior to cataract removal and a shifted anatomic location after removal of both cataract lens material and a portion of an anterior capsule, the structure being sufficiently rigid to resist deformation caused by forces acting upon the structure that are of a same magnitude and direction as that imparted by capsular contraction forces of the posterior lens capsule within the adult eye to traverse the shift distance.

20. An apparatus of claim 19, wherein the structure also has an interrupted ring configuration having two free ends separated from each other by a gap.

21. Use of an implant for manufacture of an apparatus to maintain a position of a posterior lens capsule so as to prevent an anterior shift of the posterior lens capsule, comprising
using an implant that includes an intraocular lens that has a lens optic extending within a lens optic plane, the implant having a depth that separates two parallel planes from each other;
using a structure outside of the implant and between the two parallel planes; and
using haptics that extends from the structure so as to have proximal and distal portions, the proximal portions being closer to opposite sides of the structure than are the distal portions, the distal portions being arranged within a common plane located out of the lens optic plane so that a separation distance is defined between the structure and the common plane that is substantially the same as or larger than a shift distance that would otherwise naturally occur by a posterior lens capsule within an adult eye between a normal anatomic location prior to cataract removal and a shifted anatomic location after removal of both cataract lens material and a portion of an anterior capsule, the structure being sufficiently rigid to resist deformation caused by forces acting upon the structure that are of a same magnitude and direction as that imparted by capsular contraction forces of the posterior lens capsule within the adult eye to traverse the shift distance.

22. Use of claim 21, wherein the structure also has an interrupted ring configuration having two free ends separated from each other by a gap.

23. A method of assembling an apparatus useful in maintaining a position of a posterior lens capsule so as to prevent an anterior shift of the posterior lens capsule, comprising
arranging an implant that includes an intraocular lens that has a lens optic so as to position the lens optic to extend within a lens optic plane, the implant having a depth that separates two parallel planes from each other;
arranging a structure outside of the implant and between the two parallel planes; and
arranging haptics to extend from the structure so as to have proximal and distal portions, the proximal portions being closer to opposite sides of the structure than are the distal portions, the distal portions being arranged within a common plane located out of the lens optic plane so that a separation distance is defined between the structure and the common plane that is substantially the same as or larger than a shift distance that would otherwise naturally occur by a posterior lens capsule within an adult eye between a normal anatomic location prior to cataract removal and a shifted anatomic location after removal of both cataract lens material and a portion of an anterior capsule, the structure being sufficiently rigid to resist deformation caused by forces acting upon the structure that are of a same magnitude and direction as that imparted by capsular contraction forces of the posterior lens capsule within the adult eye to traverse the shift distance.

24. A method of claim 23, wherein the structure also has an interrupted ring configuration having two free ends separated from each other by a gap.
